(19) United States
(12) Patent Application Publication
(10) Pub. No.: US 2018/0169052 A1
(43) Pub. Date: Jun. 21, 2018

(54) AGENT FOR IMPROVING BRAIN FUNCTION AND AGENT FOR PREVENTING OR TREATING COGNITIVE IMPAIRMENT

(71) Applicants: THE FOOD SCIENCE INSTITUTE FOUNDATION, Kanagawa (JP); NATIONAL CENTER OF NEUROLOGY AND PSYCHIATRY, Tokyo (JP)

(72) Inventors: Hiroshi KUNUGI, Tokyo (JP); Miho OTA, Tokyo (JP); Junko MATSUO, Tokyo (JP); Yoshitatsu ICHIHARA, Kanagawa (JP)

(73) Assignees: THE FOOD SCIENCE INSTITUTE FOUNDATION, Kanagawa (JP); NATIONAL CENTER OF NEUROLOGY AND PSYCHIATRY, Tokyo (JP)

(21) Appl. No.: 15/327,447

(22) PCT Filed: Jul. 23, 2015
(86) PCT No.: PCT/JP2015/070976

§ 371 Date: Jan. 19, 2017

(30) Foreign Application Priority Data
Jul. 23, 2014 (JP) 2014-149990

(51) Int. Cl. A61K 31/23; A61K 38/01; A61K 38/17; A61K 38/40; A61K 35/20; A61K 31/7016; A23L 33/115; A23L 33/19; A23C 9/16; A61P 25/28

(52) U.S. Cl. CPC A61K 31/23; A23V 2250/5424; A61K 38/1722; A61K 38/40; A61K 35/20; A61K 31/7016; A23L 33/115; A23L 33/19; A23C 9/16; A61P 25/28; A61K 2300/00; A23V 2002/00; A23V 2200/322; A23V 2250/1944; A61K 38/018

(57) ABSTRACT

An agent for improving brain function and agent for preventing or treating cognitive impairment, which comprises: 5 to 50 parts by weight of a protein; 5 to 80 parts by weight of a medium-chain fatty acid triglyceride as a lipid; and 1 to 50 parts by weight of a carbohydrate, per 100 parts by weight.

### TECHNICAL FIELD

The present invention relates to an agent for improving brain function and an agent for preventing or treating cognitive impairment.

### BACKGROUND ART

It is known that brain function declines with age. The number of neurons in the brain generally decreases with age.

However, when cells are lost, new connections are sometimes established among the remaining neurons, or new neurons are sometimes created in some areas of the brain even in the aged stage. Also, because cells which are more than necessary for the most of the activities are present in the brain, the decrease can be partially compensated.

On the other hand, neurons tend to lose a part of the receptors for receiving messages due to aging, and blood flow to the brain tends to decrease. Such changes due to aging decrease brain function, and thus the aged sometimes take longer time to some extent for various kinds of response or for various operations although the aged can accurately response or conduct the operations when enough time is given. Moreover, it is said that some psychic functions such as vocabulary, short-term memory, capability of learning new things and capability of remembering words decline at the age of 70 or older.

With respect to such decline in brain function, reports suggest that intake of a composition which forms a ketone body in the body is effective also against memory impairment of the aged or Alzheimer's dementia. Ketone body is a generic term for acetone, acetoacetic acid and β-hydroxybutyric acid which are produced in the liver by the metabolism of fats and released into the blood.

In general, ketone bodies are formed to compensate the energy metabolism with the ketone bodies when there is a trouble in the metabolism of glucose, for example in the case of diabetes, high fat diet, fasting (starvation), an exercise, a traumatic injury or a major operation, and the ketone bodies are used as an energy source in the skeletal muscles, the heart, the kidneys and the like. The brain usually uses glucose, which can pass the blood-brain barrier, as the energy source. When glucose is depleted by fasting or the like, acetoacetic acid and β-hydroxybutyric acid also pass the blood-brain barrier like glucose and are used as energy in the TCA cycle in the mitochondria of the cells in the brain.

Of the compositions that form a ketone body in the body, medium-chain fatty acid triglycerides are known as effective components. For example, PTL 1 describes an agent for preventing or treating Alzheimer's disease containing a medium-chain fatty acid triglyceride derived from fatty acids having 8 to 10 carbon atoms as the main component.

The estimated proportion of the patients with dementia among the aged at the age of 65 or older is 15%, and the number in 2012 is estimated at about 4,620,000. Moreover, it is estimated that there are about 4,000,000 aged people who suffer from mild cognitive impairment, which may develop to dementia. This means that one fourth of the individuals at the age of 65 or older suffer from dementia or are candidates for dementia, and it is urgent that measures against dementia should be taken in the future.

### CITATION LIST

### Patent Literature

PTL 1: JP-A-6-287138

### Non Patent Literature

NPL 1: Henderson et al (2009) Nutr Metalab. 2009; 6; 31

### SUMMARY OF INVENTION

### Technical Problem

In PTL 1, when the medium-chain fatty acid triglyceride is taken, it is necessary to consider diarrhea caused by the intake as disclosed in NPL 1.

Therefore, it is necessary to forcedly cause a medium-chain fatty acid triglyceride to be taken (administered or applied) to improve brain function, and the intake has a physical and/or psychological barrier.

Therefore, a problem to be solved by the invention is to provide a novel, safe and side-effect-free agent for improving brain function which can form a ketone body in the human body, and which does not cause diarrhea and/or nausea and is in a form that is easy to take. Also, a problem to be solved by the invention is to provide a novel, safe and side-effect-free agent for preventing or treating cognitive impairment which can form a ketone body in the human body, and which does not cause diarrhea and/or nausea and is in a form that is easy to take.

### Solution to Problem

The present inventors have conducted extensive investigation in view of the problems and made the following findings. That is, intake (administration or application) of an agent containing 5 to 50 parts by weight of a protein, 5 to 80 parts by weight of a medium-chain fatty acid triglyceride as a lipid and 1 to 50 parts by weight of a carbohydrate per 100 parts by weight promotes the formation of a ketone body in the body and thus improves brain function and can prevent or treat cognitive impairment. The problems have been thus solved.

Since the agents of the invention contain components which have been taken as food for a long time, such as a protein, a lipid and a carbohydrate, the agents of the invention are easily taken (administered or applied) continuously, are safe and carry a low risk of side effects.

Furthermore, when the agents of the invention are taken (administered or applied), a ketone body is formed in the body, and diarrhea and/or nausea are not caused. Thus, associated improvement of a symptom is expected. Examples of the subject of the intake (administration or application) include an aged person having cognitive impairment and the like. Examples of the cognitive impairment include mental retardation (dementia) as a cerebral disease, memory impairment, learning disability, consciousness disorder, cognitive disorder (agnosia), aphasia, apraxia, sleep disorder, headaches, movement disorder, sensory disorder, convulsive attack, anxiety neurosis as a mental disorder, hysteria, depression, hallucination, delusion and the like. It is believed that intake of the agents of the invention promotes the formation of a ketone body in the body and thus improves brain function and can treat the cognitive impairment.

Also, because the agents of the invention are free of side effect and safe, for example, a healthy aged person can also take the agents of the invention to improve brain function and to prevent cognitive impairment. The aged person in the invention means a person at the age of 60 or older unless otherwise specifically noted. The healthy aged person in the invention means an aged person who does not have the cognitive impairment.

That is, the invention is as follows.

[1] An agent for improving brain function, comprising: 5 to 50 parts by weight of a protein; 5 to 80 parts by weight of a medium-chain fatty acid triglyceride as a lipid; and 1 to 50 parts by weight of a carbohydrate, per 100 parts by weight.
[2] The agent for improving brain function according to the [1] above, wherein the protein is at least one kind of protein selected from the group consisting of casein, a milk protein concentrate (MPC), a whey protein concentrate (WPC), a whey protein isolate (WPI), α-lactalbumin, β-lactoglobulin and lactoferrin.
[3] The agent for improving brain function according to the [1] or [2] above which does not cause at least one of diarrhea and nausea.
[4] The agent for improving brain function according to any one of the [1] to [3] above which is for an aged person.
[5] The agent for improving brain function according to the [4] above, wherein the aged person is a healthy aged person.
[6] The agent for improving brain function according to the [4] above, wherein the aged person is an aged person having cognitive impairment.
[7] The agent for improving brain function according to any one of the [1] to [6] above which is applied in an amount of 10 g or more a day.
[8] A composition for improving brain function, comprising the agent for improving brain function according to any one of the [1] to [7] above.
[9] The composition according to the [8] above which is modified milk powder, liquid food, food for the sick, a supplement or enriched food.
[10] Use of the agent for improving brain function according to any one of the [1] to [7] above for the improvement of brain function.
[11] A method for improving brain function, comprising applying an effective amount of the agent for improving brain function according to any one of the [1] to [7] above to a subject.
[12] Use of the agent for improving brain function according to any one of the [1] to [7] above for the manufacture of a composition for improving brain function.
[13] An agent for preventing or treating cognitive impairment, comprising: 5 to 50 parts by weight of a protein; 5 to 80 parts by weight of a medium-chain fatty acid triglyceride as a lipid; and 1 to 50 parts by weight of a carbohydrate, per 100 parts by weight.
[14] The agent for preventing or treating cognitive impairment according to the [13] above, wherein the protein is at least one kind of protein selected from the group consisting of casein, a milk protein concentrate (MPC), a whey protein concentrate (WPC), a whey protein isolate (WPI), α-lactalbumin, β-lactoglobulin and lactoferrin.
[15] The agent for preventing or treating cognitive impairment according to the [13] or [14] above which does not cause at least one of diarrhea and nausea.
[16] The agent for preventing or treating cognitive impairment according to any one of the [13] to [15] above which is for an aged person.
[17] The agent for preventing or treating cognitive impairment according to the [16] above, wherein the aged person is a healthy aged person.
[18] The agent for preventing or treating cognitive impairment according to the [16] above, wherein the aged person is an aged person having cognitive impairment.
[19] The agent for preventing or treating cognitive impairment according to any one of the [13] to [18] above which is applied in an amount of 10 g or more a day.
[20] A composition for preventing or treating cognitive impairment, comprising the agent for preventing or treating cognitive impairment according to any one of the [13] to [19] above.
[21] The composition according to the [20] above which is modified milk powder, liquid food, food for the sick, a supplement or enriched food.
[22] Use of the agent for preventing or treating cognitive impairment according to any one of the [13] to [19] above for the prevention or the treatment of cognitive impairment.
[23] A method for preventing or treating cognitive impairment, comprising applying an effective amount of the agent for preventing or treating cognitive impairment according to any one of the [13] to [19] above to a subject.
[24] Use of the agent for preventing or treating cognitive impairment according to any one of the [13] to [19] above for the manufacture of a composition for preventing or treating cognitive impairment.

### Advantageous Effects of Invention

According to the invention, a novel, safe and side-effect-free agent for improving brain function and a novel, safe and side-effect-free agent for preventing or treating cognitive impairment which do not cause diarrhea and/or vomiting and which can form a ketone body in the human body in a form that is easy to take can be provided.

### BRIEF DESCRIPTION OF DRAWINGS

FIG. 1 shows the procedures of examination by the intake of the agent of the invention or a control.

FIG. 2 shows the relation between the elapsed time after the intake of the agent of the invention or the control and the blood acetoacetic acid concentration in Test Example 1.

FIG. 3 shows the relation between the elapsed time after the intake of the agent of the invention or the control and the blood β-hydroxybutyric acid concentration in Test Example 1.

FIG. 4 shows the relation between the improvement of the response time in the Trail Making Test-A after elapsed 1.5 hours and 3 hours after the intake of the agent of the invention and the blood β-hydroxybutyric acid concentration after elapsed 3 hours after the intake of the agent of the invention in Test Example 1.

FIG. 5 shows the relation between the elapsed time after the intake of the agent of the invention or the control and the blood acetoacetic acid concentration in Test Example 2.

FIG. 6 shows the relation between the elapsed time after the intake of the agent of the invention or the control and the blood β-hydroxybutyric acid concentration in Test Example 2.

### DESCRIPTION OF EMBODIMENTS

The invention is explained in detail below, but the invention is not limited to the individual embodiments.

The invention relates to an agent for improving brain function containing 5 to 50 parts by weight of a protein, 5 to 80 parts by weight of a medium-chain fatty acid triglyceride as a lipid and 1 to 50 parts by weight of a carbohydrate per 100 parts by weight or to a composition for improving brain function containing the agent for improving brain function.

The invention also relates to an agent for preventing or treating cognitive impairment containing 5 to 50 parts by weight of a protein, 5 to 80 parts by weight of a medium-chain fatty acid triglyceride as a lipid and 1 to 50 parts by weight of a carbohydrate per 100 parts by weight or to a composition for preventing or treating cognitive impairment containing the agent for preventing or treating cognitive impairment.

In this description, the “agent for improving brain function” and the “agent for preventing or treating cognitive impairment” are together referred to as “the agent of the invention”, and the “composition for improving brain function” and the “composition for preventing or treating cognitive impairment” are together referred to as “the composition of the invention”, unless otherwise specifically noted.

### <Agent of Invention>

The agent of the invention contains 5 to 50 parts by weight of a protein, 5 to 75 parts by weight of a medium-chain fatty acid triglyceride as a lipid and 1 to 50 parts by weight of a carbohydrate per 100 parts by weight.

### <Protein>

The kind of protein contained in the agent of the invention is not particularly limited, but for example, a milk protein which is a milk-derived material, such as casein, a milk protein concentrate (MPC), a whey protein concentrate (WPC), a whey protein isolate (WPI), α-lactalbumin, β-lactoglobulin and lactoferrin, and the like can be preferably used.

A kind or two or more kinds of the proteins can be used. Also, a commercial protein material can be used as long as the effects of the invention can be obtained. When the protein is used, the medium-chain fatty acid triglyceride as a lipid can be taken easily.

Moreover, protein materials which are not derived from milk, such as soy protein, rice protein, wheat protein, fish protein and meat protein, can also be used without a particular limitation as long as the effects of the invention can be obtained.

A kind or two or more kinds of the proteins can be used. Also, a commercial protein material can be used as long as the effects of the invention can be obtained. When the protein is used, the medium-chain fatty acid triglyceride as a lipid can be taken easily.

Moreover, the functional properties (physical properties such as solubility, viscosity, gelation, thermal stability and emulsion stability as well as physiological properties and the like) of the proteins may be modified according to the need through hydrolysis, modification of an amino acid residue or the like.

Furthermore, from the viewpoint of the inhibition of diarrhea, which is an effect of the invention, a fermented milk-derived protein which is expected to have an effect of regulating the gastric function and the like can also be used. As the material of the fermented milk protein, for example, a material obtained by fermenting milk with a lactic acid bacterium, a bifidobacterium and/or the like, such as yogurt or cheeses, can be used. Examples of a form of the fermented milk protein include fresh cheeses which are natural cheeses obtained by removing whey from fermented milk and which are not matured (quark, mascarpone, cream cheese and the like), and a kind or two or more kinds thereof can be used.

The amount of the protein added is 5 to 50 parts by weight per 100 parts by weight of the agent of the invention and can be appropriately adjusted within the range depending on the amounts of the other components (the medium-chain fatty acid triglyceride as a lipid and the carbohydrate), the pathological condition of the subject of the intake, the medical condition, the age, the body weight, the use or the like. The amount per 100 parts by weight of the composition is preferably 7.5 to 40 parts by weight, more preferably 10 to 30 parts by weight, further preferably 12.5 to 20 parts by weight, particularly preferably 12.5 to 17.5 parts by weight. When the protein is a hydrolysate of whey protein, in particular, the amount can be preferably applied.

### <Medium-Chain Fatty Acid Triglyceride>

The kind of medium-chain fatty acid triglyceride (MCT) as a lipid contained in the agent of the invention is not particularly restricted. As the medium-chain fatty acid triglyceride, for example, triglycerides derived from fatty acids each having 5 to 12 carbon atoms, preferably 8 to 10 carbon atoms, and the like can be preferably used. When a triglyceride having 5 to 12 carbon atoms or the like is used, for example, a ketone body can be formed efficiently in the body of a human, and the effects of the invention can be further improved. A kind or two or more kinds of the medium-chain fatty acid triglycerides can be used.

As a saturated fatty acid having 8 to 10 carbon atoms, for example, caprylic acid, capric acid and the like can be used. A commercial edible fat or oil containing a medium-chain fatty acid triglyceride (for example, product name Nisshin MCT oil (manufactured by The Nisshin OilliO Group, Ltd.)) can be used as the medium-chain fatty acid triglyceride.

The amount of the medium-chain fatty acid triglyceride added is 5 to 80 parts by weight per 100 parts by weight of the agent of the invention and can be appropriately adjusted within the range depending on the amounts of the other components (the protein and the carbohydrate), the pathological condition of the subject of the intake, the medical condition, the age, the body weight, the use or the like.

The amount per 100 parts by weight is preferably 10 to 75 parts by weight, more preferably 15 to 70 parts by weight, further preferably 20 to 65 parts by weight, further preferably 25 to 60 parts by weight, further preferably 30 to 55 parts by weight, further preferably 30 to 50 parts by weight, particularly preferably 35 to 45 parts by weight.

### <Carbohydrate>

The kind of carbohydrate contained in the agent of the invention is not particularly restricted. From the viewpoint of not increasing the blood sugar level after the intake, as the carbohydrate, for example, lactose, palatinose, trehalulose and the like can be preferably used. When the carbohydrate is used, the medium-chain fatty acid triglyceride as a lipid can be taken easily. Also, to further improve the effects of the invention, a carbohydrate material which is expected to have an action of regulating the gastric function, such as dietary fiber, can be used. A kind or two or more kinds of the carbohydrates can be used.

The amount of the carbohydrate added is 1 to 50 parts by weight per 100 parts by weight of the agent of the invention and can be appropriately adjusted within the range depending on the amounts of the other components (the protein and the medium-chain fatty acid triglyceride as a lipid), the pathological condition of the subject of the intake, the medical condition, the age, the body weight, the use or the like. The amount per 100 parts by weight of the agent of the invention is preferably 2 to 40 parts by weight, more preferably 3 to 30 parts by weight, further preferably 4 to 20 parts by weight, further preferably 5 to 16 parts by weight, further preferably 6 to 14 parts by weight, further preferably 7 to 12 parts by weight, particularly preferably 8 to 10 parts by weight.

### <Other Components>

The agent of the invention can contain another fat or oil material as a lipid other than the above lipid, and the source and the kind thereof are not restricted. For example, a saturated fatty acid (palmitic acid, stearic acid or the like), a monovalent unsaturated fatty acid (oleic acid or the like), a polyvalent unsaturated fatty acid (linoleic acid, linolenic acid or the like), a phospholipid and the like can be used. Also, a long-chain fatty acid oil and fat (LCT) and the like can also be used.

As the phospholipid material, for example, one, two or more kinds of known phospholipid materials such as a milk phospholipid, soy lecithin and egg yolk lecithin can be used.

The phospholipid can be fractionated or purified from a source material such as milk, soybean, egg or the like. A commercial material containing a phospholipid can be used as long as the effects of the invention can be obtained.

The amount of the phospholipid added can be appropriately adjusted depending on the amounts of the other components (the protein, the medium-chain fatty acid triglyceride as a lipid and the carbohydrate), the pathological condition of the subject of the intake, the medical condition, the age, the body weight, the use or the like. For example, the amount of the milk phospholipid added is 0.01 to 1 part by weight per 100 parts by weight of the agent of the invention, preferably 0.05 to 0.8 parts by weight, more preferably 0.1 to 0.7 parts by weight, further preferably 0.15 to 0.6 parts by weight, further preferably 0.2 to 0.5 parts by weight, further preferably 0.25 to 0.45 parts by weight, particularly preferably 0.3 to 0.4 parts by weight.

With respect to the other fat or oil material, the amounts of a saturated fatty acid (palmitic acid, stearic acid or the like), a monovalent unsaturated fatty acid (oleic acid or the like) and a polyvalent unsaturated fatty acid (linoleic acid, linolenic acid or the like) added can be adjusted by comparing the standard of the intake of lipids set by the Ministry of Health, Labour and Welfare and the actual intake of lipids. Of the lipids, it is difficult to increase the amount of intake of a monovalent unsaturated fatty acid by the diet only in this country, and thus it is also preferable to increase the proportion of a monovalent unsaturated fatty acid in all the fatty acids.

Examples of the monovalent unsaturated fatty acid include oleic acid, palmitoleic acid, myristoleic acid and the like. Examples of a lipid source containing a high amount of oleic acid include high oleic sunflower oil containing a high amount of oleic acid, rapeseed oil, olive oil, high oleic safflower oil, soybean oil, corn oil, palm oil and the like. Also, a lipid source containing oleic acid is nutrient-modified fat or oil (manufactured by Nippon Oil & Fats Co., Ltd.). Moreover, sunflower oil, rapeseed oil, olive oil and a mixture with olive oil can also be used.

The amount of the other fat or oil material added can be appropriately adjusted depending on the amounts of the other components (the protein, the medium-chain fatty acid triglyceride as a lipid and the carbohydrate), the pathological condition of the subject of the intake, the medical condition, the age, the body weight, the use or the like. For example, the amount of high oleic sunflower oil containing a high amount of oleic acid added is 0 to 60 parts by weight per 100 parts by weight of the agent of the invention, preferably 5 to 55 parts by weight, more preferably 10 to 50 parts by weight, further preferably 15 to 45 parts by weight, further preferably 20 to 40 parts by weight, particularly preferably 25 to 35 parts by weight.

In addition, as materials of the agent of the invention, known food and food additive can be added for the purpose of making it easy to take the agent safely without a side effect. Moreover, for the purpose of improving the effects of the invention safely without a side effect, food and a food additive which improve the intestinal flora and which can inhibit diarrhea, such as known dietary fiber, can be added. Furthermore, for the purpose of improving the effects of the invention, food and a food additive which improve the flavor so that the agent becomes easy to take and which can inhibit vomiting can be added.

In addition to the protein, the lipid, the carbohydrate and the like, water and a known material that a human can take (that can be administered or applied to a human) can be added to the agent of the invention. For example, from the viewpoint of inhibiting a side effect, a food material, a food additive and the like which have been often used for food can be used. Moreover, the agent of the invention is in any form such as liquid, solid, powder or gel. The agent of the invention can be prepared by a known production method.

### <Method for Using Agent of Invention>

By applying an effective amount to a subject, the agent of the invention can exhibit the effect of improving brain function or the effect of preventing or treating cognitive impairment. Even by single intake (single administration), the agent of the invention is expected to exhibit the effect. In the case of a human of a body weight of 60 kg for example, the effective amount thereof as powder is 10 to 90 g a day, preferably 15 to 85 g, more preferably 20 to 80 g, further preferably 25 to 75 g, further preferably 30 to 70 g, further preferably 35 to 65 g, further preferably 40 to 60 g, particularly preferably 45 to 55 g. When the agent of the invention is taken (administered or applied), the powder which is dispersed or dissolved in an adequate amount of water in advance can be used. Moreover, the agent of the invention can be taken (administered or applied) by a known method such as an oral, transluminal or enteral method.

Because the agent of the invention does not cause a side effect such as diarrhea and/or nausea, the agent of the invention can be taken continuously, and a higher effect thereof is expected. In the case of a human of a body weight of 60 kg for example, the effective amount thereof as powder is the intake (administration or application) of 10 g or more a day, 15 g or more a day, 20 g or more a day, 25 g or more a day, 30 g or more a day, 35 g or more a day, 40 g or more a day, 45 g or more a day or 50 g or more a day. Also, in the case of a human of a body weight of 60 kg for example, the intake (administration or application) is for one day or longer, two days or longer, three days or longer, four days or longer, five days or longer, six days or longer, one week or longer, two weeks or longer, three weeks or longer, four weeks or longer, six weeks or longer, eight weeks or longer, 12 weeks or longer or 24 weeks or longer. Moreover, for example, continuous intake is once a week, twice a week, three times a week, four times a week, five times a week, six times a week or seven times a week. In particular, application of 10 g or more a day is preferable.

Examples of the subject of the intake (administration or application) of the agent of the invention include an aged person, a patient with dementia, a patient with epilepsy, an adult and the like. The agent of the invention can be taken by an aged person even when the aged person is an aged person having cognitive impairment or a healthy aged person.

Examples of the cognitive impairment include mental retardation (dementia) as a cerebral disease, memory impairment, learning disability, consciousness disorder, cognitive disorder (agnosia), aphasia, apraxia, sleep disorder, headaches, movement disorder, sensory disorder, convulsive attack, anxiety neurosis as a mental disorder, hysteria, depression, hallucination, delusion and the like. The agent of the invention can be used for an aged person having the cognitive impairment and can improve brain function or treat the cognitive impairment.

Because the agent of the invention is free of side effect and safe, the agent of the invention can be used also as an agent for preventing cognitive impairment for a healthy aged person.

### <Composition of Invention>

The agent of the invention can be applied alone but can also be used as a composition containing the agent of the invention (the composition of the invention). That is, the agent of the invention can be used for the manufacture of the composition of the invention. The composition of the invention can be used for the improvement of brain function or for the prevention or the treatment of cognitive impairment.

When the composition of the invention is produced, the amount of the agent of the invention added to the composition can be optionally determined depending on the purpose, the use, the form, the dosage form, the symptom, the body weight and the like. Although the invention is not limited thereto, as the amount thereof relative to the total amount, the agent of the invention can be added in an amount of 5 to 95% (w/w) and can be added further preferably in an amount of 10 to 50% (w/w). This is because the intake (administration or application) becomes easy in the range.

Specifically, the composition of the invention can be used both in the form of a pharmaceutical product (a pharmaceutical composition) and in the form of food or drink (a food or drink composition). For example, the composition of the invention is expected to exhibit the effect of improving brain function or the effect of preventing or treating cognitive impairment by directly administering the composition as a pharmaceutical product or by directly taking the composition as food for special dietary uses such as foods for specified health uses or nutritional food. Examples of the food for special dietary uses and the nutritional food include modified milk powder, liquid food, food for the sick, a supplement, enriched food and the like.

When the composition of the invention is used as a pharmaceutical composition, the administration is for example oral administration in the form of pharmaceutical preparation such as tablets, coated tablets, capsules, granules, powder, solutions, syrup or emulsions. The composition of the invention can be obtained by preparing a pharmaceutical preparation of the agent of the invention as the main drug according to a normal method using a known adjuvant which can be generally used in the field of pharmaceutical formulations, such as a dispersant, an excipient, a binder, a disintegrant, a lubricant, a colorant, a flavoring agent, a solubilizer, a suspending agent and a coating agent.

Examples of the dispersant include milk proteins such as casein, soy protein, peptides, amino acids, starch, dextrin, xylan, oligosaccharides, saccharides (glucose, lactose, sucrose, galactose and maltose), sugar alcohols (trehalose, xylitol, erythritol, palatinose, trehalulose and xylose) and the like.

Examples of the excipient include lactose, fructose, glucose, cornstarch, sorbitol, crystalline cellulose and the like.

Examples of the binder include methyl cellulose or a salt thereof, ethyl cellulose, gum arabic, gelatin, hydroxypropyl cellulose, polyvinylpyrrolidone and the like.

Examples of the disintegrant include starch, sodium alginate, gelatin, calcium carbonate, calcium citrate, dextrin, magnesium carbonate, synthetic magnesium silicate and the like.

Examples of the lubricant include talc, magnesium stearate, polyethylene glycol, hydrogenated vegetable oil and the like.

Examples of the colorant include colorants which are approved as additives for pharmaceutical products, and cocoa powder, menthol, aromatic powder, mint oil, borneol, cinnamon powder or the like is used as the flavoring agent.

Examples of the solubilizer include polyvinylpyrrolidone, sorbitan monostearate, sorbitan monopalmitate, sorbitan monolaurate, polyvinyl alcohol and the like.

When the composition of the invention is prepared as a food or drink composition without a side effect, the agent of the invention may be added to various foods and drinks (cow's milk, soft drinks, fermented milk, yogurt, cheeses, breads, biscuits, crackers, pizza crust, modified milk powder, liquid food, food for the sick, nutritional food and the like) and then taken. The agent of the invention can be used according to a normal method for general food or drink compositions, for example by directly using the agent of the invention or mixing with other food or food components.

The state thereof may be any generally used state of food or drink such as solid (powder, granules and the like), paste, liquid or suspension. In such a form, the composition of the invention can be taken without a psychological problem.

When the composition of the invention is provided as a food composition or a pharmaceutical composition, the production method thereof may be a method known to one skilled in the art. One skilled in the art can produce a desired food or pharmaceutical preparation by appropriately combining a step of mixing the agent of the invention with other components, a forming step, a sterilization step, a fermentation step, a calcination step, a drying step, a cooling step, a granulation step, an enclosing step and the like.

The composition of the invention can be applied also to food with health claims or food for the sick. The system for food with health claims has been established not only for general foods but also for foods in the form of tablets, capsules and the like, in view of the trends inside and outside of Japan and also considering the consistency with the existing system for foods for specified health uses. The system for food with health claims includes two types, namely foods for specified health uses (individual approval system) and foods with nutrient function claims (standard regulation system). It is expected that the effect of improving brain function or the effect of preventing or treating cognitive impairment is exhibited when the composition of the invention is directly taken as food for special dietary uses such as foods for specified health uses or food with nutrient function claims.

The subject of the intake (administration or application) of the composition of the invention is similar to that of the agent of the invention, and examples thereof include an aged person, a patient with dementia, a patient with epilepsy, an adult and the like. The composition of the invention can be taken by an aged person even when the aged person is an aged person having cognitive impairment or a healthy aged person.

Examples of the cognitive impairment include mental retardation (dementia) as a cerebral disease, memory impairment, learning disability, consciousness disorder, cognitive disorder (agnosia), aphasia, apraxia, sleep disorder, headaches, movement disorder, sensory disorder, convulsive attack, anxiety neurosis as a mental disorder, hysteria, depression, hallucination, delusion and the like. The composition of the invention can be used for an aged person having the cognitive impairment and can improve brain function or treat the cognitive impairment.

Because the composition of the invention is free of side effect and safe, the composition of the invention can be used also for the prevention of cognitive impairment for a healthy aged person for example.

### EXAMPLES

The invention is explained in further detail below referring to Examples, but the invention is not limited by the Examples.

### (Test Example 1) Experiment 1 of Administration of Agent of Invention to Healthy Aged Individuals

### [Production of Agent of Invention]

Powder containing a medium-chain fatty acid triglyceride (MCT) was obtained by blending a composition containing 40% by weight of the medium-chain fatty acid triglyceride, 30.1% by weight of a long-chain fatty acid oil and fat (LCT), 0.4% by weight of soy lecithin, 8% by weight of lactose, 0.2% by weight of citric acid, 14% by weight of casein, 4% by weight of a whey protein concentrate 0.2% by weight of vitamins and 3.1% by weight of minerals with water for dissolution, emulsifying the solution by homogenization and spray drying the solution. A solution obtained by dissolving 50 g of the powder in 79 g of water (total 129 g) was used as “the agent of the invention” below.

As a control, 825 g of vegetable fat cream for whipping (“Whip” manufactured by Megmilk Snow Brand Co., Ltd.), 400 g of cow's milk and 62.5 g of protein powder (“Meiji Mei Protein Zn” manufactured by Meiji Co., Ltd.) were mixed, blended and emulsified by homogenization. The obtained material was used as “the control” below.

### [Selection of Subjects]

Of the individuals at the age of 60 or older who were recruited using a website, leaflets and the like, two males and five females who gave written consent (the average age was 65.9±3.1 years old) were selected as subjects. The seven subjects did not have any medical diseases such as impaired liver/kidney function, hyperlipidemia and diabetes and did not have any organic brain disease. The seven subjects consulted with a doctor psychiatrically as to whether the subjects had cognitive impairment, and it was confirmed that the subjects did not suffer from any mental diseases.

### [Intake of Agent of Invention]

A crossover study was conducted using “the agent of the invention” and “the control” using the seven subjects, and the increases in the blood ketone body concentrations and the effect of improving cognitive function were examined.

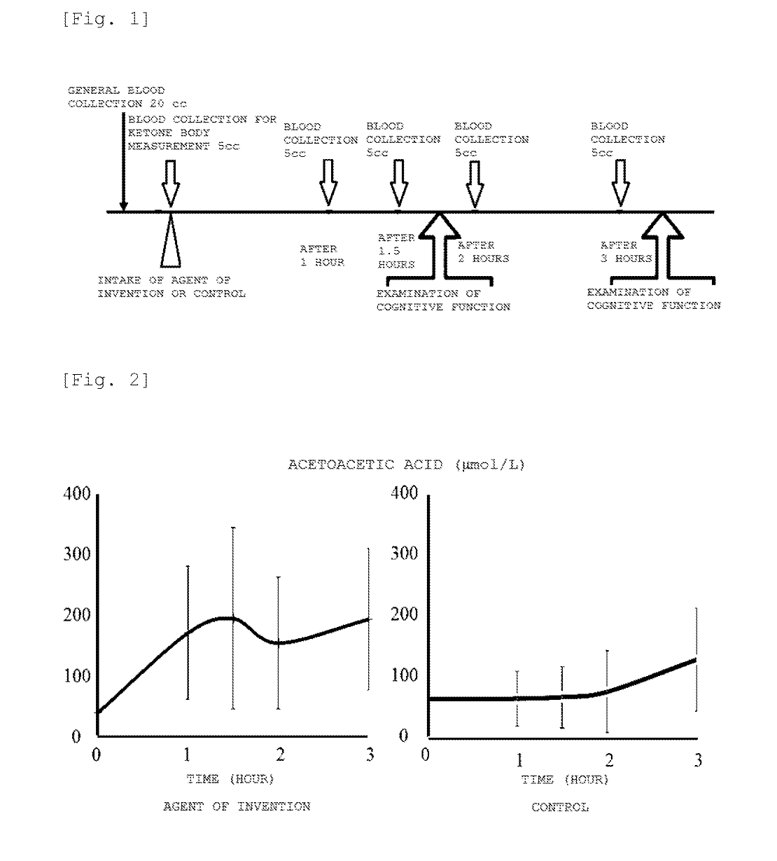

Specifically, first, the subjects fasted after 22 o'clock on the day before the examination, and the examination was started at 9 o'clock on the day of the examination. Blood samples were collected from the vein, and the liver function, the kidney function, the fasting blood sugar level, the cholesterol value and the like were evaluated. At 10 o'clock on the same day, 129 g of the agent of the invention produced above (the solution obtained by dissolving 50 g of the powder (containing 20 g of the medium-chain fatty acid) in 79 g of water) was taken once, and 129 g of the control (corresponding to one in which the medium-chain fatty acid component was replaced with another fatty acid) was taken once on another day. The order of “the agent of the invention” and “the control” was determined by a simple randomized method. Blood samples were collected from the vein 1, 1.5, 2 and 3 hours after the start of the intake, and the blood ketone body concentrations (acetoacetic acid and β-hydroxybutyric acid in the blood) were measured. The cognitive function was evaluated 1.5 hours and 3 hours after the intake by the Trail Making Test (document: Lezak M D, Howieson D B, Loring D W. Neuropsychological Assessment. NY Oxford University Press 2004.), which examines maintenance of attention, selection and visual search/visual-motor coordination (FIG. 1). The subjects were asked about any adverse events such as diarrhea and nausea during the examination.

### [Evaluation]

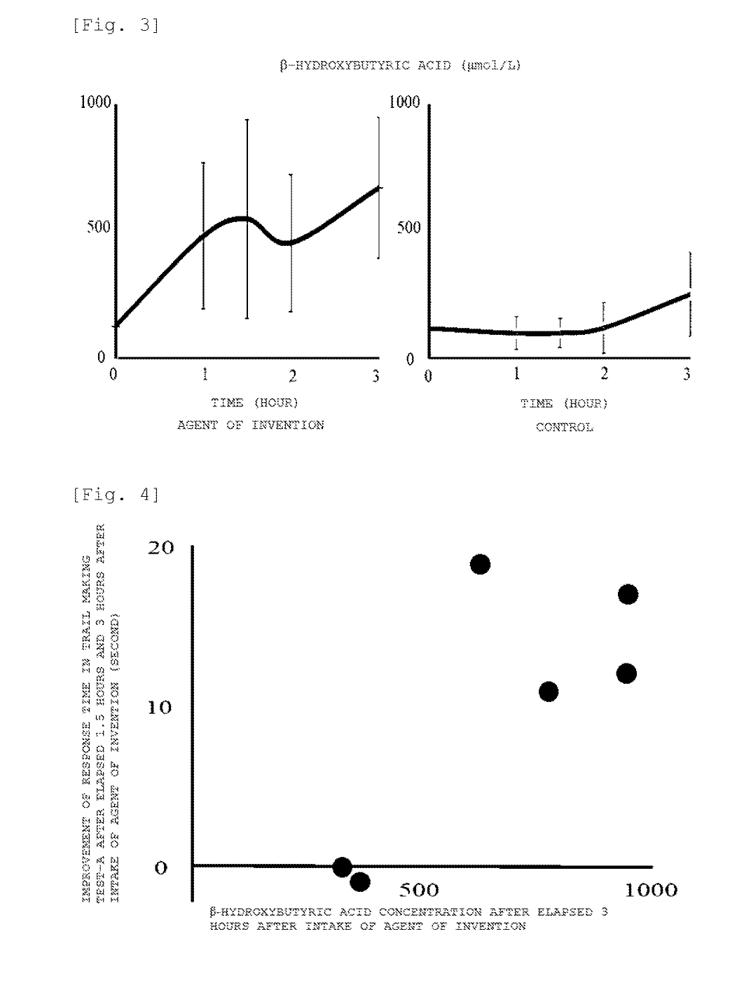

As a result of the measurement of the blood ketone body concentrations, it was confirmed that the acetoacetic acid concentration (FIG. 2) and the β-hydroxybutyric acid concentration (FIG. 3) in the blood increased with time after the intake of “the agent of the invention” as compared to the concentrations after the intake of “the control”.

When the scores of the Trail Making Test-A were examined, in the case of the intake of “the control”, the average response times were 29.0±7.5 seconds 1.5 hours after the intake of the control and 29.1±13.6 seconds 3 hours after the intake of the control, and an effect of the intake of the control (a decrease in the average response time) was not observed.

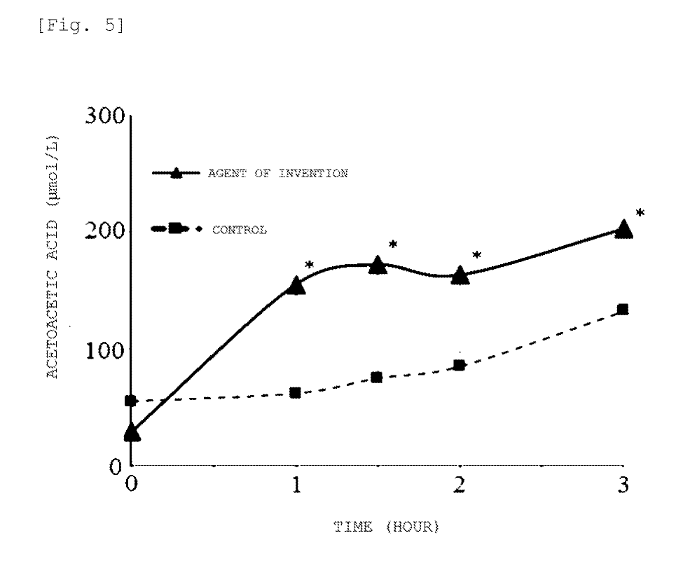

On the other hand, in the case of the intake of “the agent of the invention”, the average response times were 34.6±7.8 seconds 1.5 hours after the intake of the agent of the invention and 25.1±4.7 seconds 3 hours after the intake of the agent of the invention. An effect of the intake of the agent of the invention (a decrease in the average response time) was observed, and the scores of the Trail Making Test-A improved as the blood ketone body concentrations increased (FIG. 4).

The above results suggest that the agent of the invention has the action of improving cognitive function in healthy aged individuals. In this regard, no serious side effect was observed in the series of examination. Transient nausea was observed after the intake of the agent of the invention in only one case of the seven cases, but the symptom disappeared quickly. In any of the seven cases, symptom of diarrhea was not observed after the intake of the agent of the invention.

### (Test Example 2) Experiment 2 of Administration of Agent of Invention to Healthy Aged Individuals

### [Production of Agent of Invention]

“The agent of the invention” and “the control” were produced in similar manners to those of Test Example 1.

### [Selection of Subjects]

Of the individuals at the age of 60 or older who were recruited using a website, leaflets and the like, six males and 14 females who gave written consent (the average age was 66.3±2.9 years old) were selected as subjects. The 20 subjects did not have any medical diseases such as impaired liver/kidney function, hyperlipidemia and diabetes and did not have any organic brain disease. The 20 subjects consulted with a doctor psychiatrically as to whether the subjects had cognitive impairment, and it was confirmed that the subjects did not suffer from any mental diseases.

### [Intake of Agent of Invention]

In a similar manner to that of Test Example 1, a crossover study was conducted using “the agent of the invention” and “the control” using the subjects, and the increases in the blood ketone body concentrations and the effect of improving cognitive function were examined. One and a half hours and three hours after the intake of “the agent of the invention” or “the control”, the cognitive function was evaluated by the Trail Making Test (document: Lezak M D, Howieson D B, Loring D W. Neuropsychological Assessment. NY Oxford University Press 2004.), which examines maintenance of attention, selection and visual search/visual-motor coordination, and the concentration and the memory retention were evaluated by the digit span assessment and the visual memory span assessment of the Japanese-version Wechsler Memory Scale (document: Wechsler D. Wechsler memory scale-revised. 1987; San Antonio; Psychological Corporation.) (FIG. 1). The subjects were asked about any adverse events such as diarrhea and nausea during the examination.

### [Evaluation]

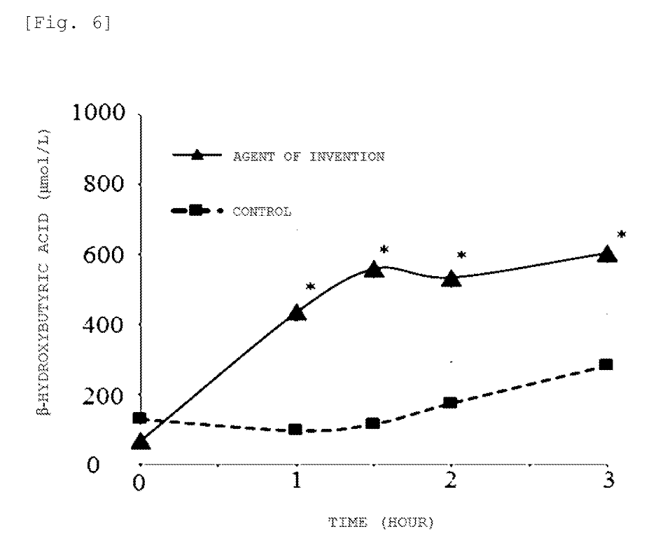

As a result of the measurement of the blood ketone body concentrations, it was confirmed that the acetoacetic acid concentration (FIG. 5) and the β-hydroxybutyric acid concentration (FIG. 6) in the blood increased with time after the intake of “the agent of the invention” as compared to the concentrations when “the control” was taken.

When the scores of the Trail Making Test-B were examined, in the case of the intake of “the control”, the average response times were 77.8±23.7 seconds 1.5 hours after the intake of the control and 79.0±28.9 seconds 3 hours after the intake of the control, and an effect of the intake of the control (a decrease in the average response time) was not observed.

On the other hand, in the case of the intake of “the agent of the invention”, the average response times were 75.0±18.9 seconds 1.5 hours after the intake of the agent of the invention and 70.7±26.6 seconds 3 hours after the intake of the agent of the invention. An effect of the intake of the agent of the invention (a decrease in the average response time) was observed, and the scores of the Trail Making Test-B improved as the blood ketone body concentrations increased.

Moreover, when the scores of the digit span assessment of the Japanese-version Wechsler Memory Scale were examined, the average forward and backward digit span in the case of the intake of “the control” was 11.6±3.5 digits 1.5 hours after the intake of the control, but the average forward and backward digit span in the case of the intake of “the agent of the invention” was 12.5±3.2 digits 1.5 hours after the intake of the agent of the invention. Thus, significant improvement was observed.

As the results of Test Example 1, the above results also suggest that the agent of the invention has the action of improving cognitive function in healthy aged individuals. In this regard, no serious side effect was observed in the series of examination. In any of the 20 cases, symptom of diarrhea was not observed after the intake of the agent of the invention.

### (Test Example 3) Experiment of Administration of Agent of Invention to Patients with Dementia

### [Production of Agent of Invention]

“The agent of the invention” and “the control” were produced in similar manners to those of Test Example 1.

### [Selection of Subjects]

In this test, two patients with dementia (Subject 1, a 70-year-old male, and Subject 2, a 67-year old male) were selected as the subjects.

### [Intake of Agent of Invention by Subjects]

In a similar manner to that of Test Example 1, a crossover study was conducted using “the agent of the invention” and “the control” using the subjects, and the blood ketone body concentrations were measured. The specific method for taking “the agent of the invention” or “the control” was similar to that of Test Example 1. Blood samples were collected from the vein before the intake of “the agent of the invention” or “the control” and two hours after the intake, and the blood ketone body concentrations (acetoacetic acid, β-hydroxybutyric acid and the total of the ketone bodies in the blood) were measured.

### [Evaluation]

The measurement results of Subject 1 are shown in Table 1, and the measurement results of Subject 2 are shown in Table 2.

In both subjects, it was confirmed that the acetoacetic acid concentration, the β-hydroxybutyric acid concentration and the total ketone body concentration in the blood increased significantly after the intake of “the agent of the invention” as compared to the concentrations after the intake of “the control”. The results suggested that “the agent of the invention” is effective for preventing or treating cognitive impairment such as dementia. In this regard, no serious side effect was observed in the series of examination. In either of the two cases, symptom of diarrhea was not observed after the intake of the agent of the invention.

TABLE 1

| | | β- | Total of |
|---|---|---|---|
| | Acetoacetic | hydroxybutyric | ketone |
| | acid | acid | bodies |
| | (μmol/L) | (μmol/L) | (μmol/L) |
| Before the intake of the | 42 | 93 | 135 |
| agent of the invention | | | |
| Two hours after the intake of | 86 | 330 | 416 |
| the agent of the invention | | | |
| Before the intake of the | 28 | 57 | 85 |
| control | | | |
| Two hours after the intake of | 14 | 23 | 37 |
| the control | | | |

TABLE 2

| | | β- | Total of |
|---|---|---|---|
| | Acetoacetic | hydroxybutyric | ketone |
| | acid | acid | bodies |
| | (μmol/L) | (μmol/L) | (μmol/L) |
| Before the intake of the | 35 | 43 | 78 |
| agent of the invention | | | |
| Two hours after the intake of | 87 | 271 | 358 |
| the agent of the invention | | | |
| Before the intake of the | 3 | 51 | 54 |
| control | | | |
| Two hours after the intake of | 8 | 92 | 100 |
| the control | | | |

### INDUSTRIAL APPLICABILITY

When the agent of the invention is taken (administered or applied), a ketone body is formed in the body, and diarrhea and/or nausea are not caused. Thus, associated improvement of a symptom is expected. An example is the improvement of cognitive function of an aged person, such as mental retardation (dementia) as a cerebral disease, memory impairment, learning disability, consciousness disorder, cognitive disorder (agnosia), aphasia, apraxia, sleep disorder, headaches, movement disorder, sensory disorder, convulsive attack, anxiety neurosis as a mental disorder, hysteria, depression, hallucination or delusion. Moreover, because the agent of the invention is free of side effect and safe, the agent of the invention can be taken also as an agent for preventing dementia for a healthy aged person for example.

Although the invention has been explained in detail using specific embodiments, it is obvious to one skilled in the art that various changes and modifications can be made without departing from the purpose and the scope of the invention. The present application is based on a Japanese patent application filed on Jul. 23, 2014 (patent application No. 2014-149990), which is hereby incorporated by reference in its entirety.

1. An agent for improving brain function, comprising: 5 to 50 parts by weight of a protein; 5 to 80 parts by weight of a medium-chain fatty acid triglyceride as a lipid; and 1 to 50 parts by weight of a carbohydrate, per 100 parts by weight.

2. The agent for improving brain function according to claim 1, wherein the protein is at least one kind of protein selected from the group consisting of casein, a milk protein concentrate (MPC), a whey protein concentrate (WPC), a whey protein isolate (WPI), α-lactalbumin, β-lactoglobulin and lactoferrin.

3. The agent for improving brain function according to claim 1 which does not cause at least one of diarrhea and nausea.

4. The agent for improving brain function according to claim 1 which is for an aged person.

5. The agent for improving brain function according to claim 4, wherein the aged person is a healthy aged person.

6. The agent for improving brain function according to claim 4, wherein the aged person is an aged person having cognitive impairment.

7. The agent for improving brain function according to claim 1 which is applied in an amount of 10 g or more a day.

8. A composition for improving brain function, comprising the agent for improving brain function according to claim 1.

9. The composition according to claim 8 which is modified milk powder, liquid food, food for the sick, a supplement or enriched food.

10. Use of the agent for improving brain function according to claim 1 for the improvement of brain function.

11. A method for improving brain function, comprising applying an effective amount of the agent for improving brain function according to claim 1 to a subject.

12. Use of the agent for improving brain function according to claim 1 for the manufacture of a composition for improving brain function.

13. An agent for preventing or treating cognitive impairment, comprising: 5 to 50 parts by weight of a protein; 5 to 80 parts by weight of a medium-chain fatty acid triglyceride as a lipid; and 1 to 50 parts by weight of a carbohydrate, per 100 parts by weight.

14. The agent for preventing or treating cognitive impairment according to claim 13, wherein the protein is at least one kind of protein selected from the group consisting of casein, a milk protein concentrate (MPC), a whey protein concentrate (WPC), a whey protein isolate (WPI), α-lactalbumin, β-lactoglobulin and lactoferrin.

15. The agent for preventing or treating cognitive impairment according to claim 13 which does not cause at least one of diarrhea and nausea.

16. The agent for preventing or treating cognitive impairment according to claim 13 which is for an aged person.

17. The agent for preventing or treating cognitive impairment according to claim 16, wherein the aged person is a healthy aged person.

18. The agent for preventing or treating cognitive impairment according to claim 16, wherein the aged person is an aged person having cognitive impairment.

19. The agent for preventing or treating cognitive impairment according to claim 13 which is applied in an amount of 10 g or more a day.

20. A composition for preventing or treating cognitive impairment, comprising the agent for preventing or treating cognitive impairment according to claim 13.

21. The composition according to claim 20 which is modified milk powder, liquid food, food for the sick, a supplement or enriched food.

22. Use of the agent for preventing or treating cognitive impairment according to claim 13 for the prevention or the treatment of cognitive impairment.

23. A method for preventing or treating cognitive impairment, comprising applying an effective amount of the agent for preventing or treating cognitive impairment according to claim 13 to a subject.

24. Use of the agent for preventing or treating cognitive impairment according to claim 13 for the manufacture of a composition for preventing or treating cognitive impairment.
